SEQUENCE LISTING (1 sequence)

SEQ ID NO: 1 - protein, 25 aa, ARTIFICIAL SEQUENCE
  CYS-CYS-HIS-HIS ZINC FINGER
YQCEICGKSFSDKSNLTRHLRIHTG

(19) United States
(12) Patent Application Publication
(10) Pub. No.: US 2002/0155576 A1
(43) Pub. Date: Oct. 24, 2002

(54) Metal-chelated nucleic acid binding peptides for in vivo detection and therapy of disease

(76) Inventors: Stanley L. Mills, Goldsby, OK (US); Jacqueline L. Mills, (US)

Correspondence Address: DUNLAP, CODDING & ROGERS P.C., PO BOX 16370, OKLAHOMA CITY, OK, 73114, US

(21) Appl. No.: 09/774,940

(22) Filed: Jan. 31, 2001

Related U.S. Application Data
(63) Continuation of application No. 09/021,085, filed on Feb. 10, 1998, now abandoned.

(51) Int. Cl. A61K 51/00; C12N 9/64; C07K 16/46

(52) U.S. Cl. 435/226; 530/391.1; 424/1.49

(57) ABSTRACT

The present invention relates to the diagnosis and treatment of diseases such as heart disease and cancer wherein necrosis is a part of the standard course of the disease. The method uses zinc finger proteins and their analogues having a metal chelated thereto, providing appropriate conformation for binding to DNA in necrotic tissue. Medically useful metal ions such as radioisotopes and nuclear magnetic resonance enhancing metals are attached to the zinc fingers.

As a diagnostic tool the uptake of this new class of radiopharmaceuticals pre and post conventional cancer therapy can provide almost instantaneous determination of effectiveness of the therapy and the extent of normal healthy tissue destruction. As a nuclear medicine diagnostic tool in cancer it can provide rapid prognosis and extent of disease on a physiological basis rather than conventional anatomy analysis by computerized tomography (CT) or magnetic resonance imaging (MRI). As a MRI contrast agent it can provide clear distinctions between normal tissue (no uptake) and diseased tissue (uptake). As a therapeutic agent for cancer, the compound bound to DNA in necrotic cells in the layer below the rapidly proliferating layer will irradiate the rapidly growing rim of cancerous cells with beta or alpha radiation.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

The present invention relates to therapeutic and detection methods using metal chelated peptides which can bind to cellular DNA and/or RNA in necrotic or damaged tissues.

Cancer has few unique features which can be exploited to a positive advantage. Current treatments are plagued with side effects and poor outcomes offering only limited long term survival. One pathological trait varying from normal tissue actually affords the tumor with improved survival over normal health tissue. The basic construction and rapid growth of tumors require vascular proliferation, resulting in vascular levels of up to double surrounding normal tissues. These new vessels typically have walls constructed of a single endothelial layer rather than construction typical of normal blood vessels. As the tumor grows the resultant mass compresses the vessel and restricts the nutrient supply causing some of the interior cells to become necrotic. As these cells die there is an irregular clumping of chromatin, marked swelling of organelles and focal disruption of membranes that subsequently disintegrate. The disruption of the membranes provide a unique marker, DNA, unavailable in the intact normal cell which can be exploited to a positive outcome.

Among the most common DNA-binding motifs in eukaryotic cells is the zinc finger. Between 0.5% and 1% of the human genome codes for zinc finger motifs in perhaps hundreds or more different proteins. Zinc fingers have been found to play pivotal roles in the control of eukaryotic gene transcription. To date there are three families of zinc fingers based upon the amino acids which bind to the zinc atom to form DNA binding conformations. These are the Cys-Cys-His-His family, the Cys-Cys-Cys-Cys family, and the Cys-Cys-His-Cys family. An example of the Cys-Cys-Cys-Cys family is the DNA-binding domain of the two zinc fingers (residues 440-510) of the glucocorticoid receptor. An example of the Cys-Cys-His-His is the first of nine zinc fingers (residues 13-37) that control the transcription of the 5S RNA gene in Xenopus oocytes (TFIIIA). An example of Cys-Cys-His-Cys family is the DNA-binding gag protein p55 of retrovirus HIV. These examples and many others are well known to persons of ordinary skill in the art and require no further explanation herein.

It is an object of the present invention to use zinc finger and zinc finger analogues to serve as carriers of medically useful metals either radioactive or non-radioactive to sites of tissue abnormalities for diagnostic purposes.

It is a further object of the present invention to use zinc finger and zinc finger analogues to carry medically useful metals either radioactive or non-radioactive to sites of tissue abnormalities for therapeutic purposes.

### SUMMARY OF THE INVENTION

The present invention provides drug compositions for delivery of radioactive and non-radioactive metals to sites of cellular necrosis and/or apoptosis. The drug compositions comprise zinc fingers and/or zinc finger analogues wherein the zinc is substituted by another metal (or radioactive zinc) and wherein the substituted zinc fingers or analogues have the capability of binding to DNA. The preferential uptake in the diseased tissue over normal tissue has always been a limiting step in the diagnosis and treatment of cancer. Normal tissue with intact membranes will exclude the metal labeled zinc finger analogues while diseased tissue having cells with damaged membranes allows the entry of the zinc fingers. Therefore the preferential uptake of the zinc fingers and attachment to DNA therein occurs in a manner not seen in previous agents. Accordingly, the present invention is directed to compositions containing a peptide or peptides capable of binding to DNA and having a metal incorporated therein to achieve the conformation necessary for this binding. Administration of an effective dose of such a composition to a recipient can provide diagnostic information about the disease or level or lack of disease. Administration of an effective dose of such a composition to a recipient can provide therapeutic treatments of a disease in the recipient.

### DETAILED DESCRIPTION OF THE INVENTION

Normal cell membranes resist the transfer of DNA binding proteins such as zinc fingers from the intracellular space to the extracellular space, and uptake from the extracellular space to the intracellular space. Only cells with abnormal cell membranes such as necrotic and/or apoptotic cells generally allow DNA binding proteins and peptides such as zinc fingers to be taken up from the extracellular space. Necrosis and/or apoptosis is a common occurrence in diseases like cancer and infarcted myocardium. Furthermore, these proteins require metal chelation to produce the binding conformation necessary to allow attachment to the DNA. In a degenerating cell, a zinc finger having a metal chelated thereto will be tightly bound to DNA therein for an extended period of time. Where used herein the term “zinc finger” is intended to refer to a peptide or polypeptide comprising four cysteine (Cys) residues, or three Cys residues and one histidine (His) residue, or two Cys and two His residues in such a sequence that a zinc-substituting metal can be bound thereto via the binding residues. That is, the “zinc finger” is the peptide backbone for the entire metal-peptide compound.

When the metal label of the zinc finger is a radioactive metal such as ⁶⁵Zn, ^{99m}Tc, and ⁹⁷Ru, the zinc finger would be used to identify location of tumors by external imaging with standard nuclear medicine imaging equipment. When the metal label of the zinc finger is a nonradioactive metal such as gadolinium (Gd) or manganese (Mn), the zinc finger would be used to identify size and location of tumors by magnetic resonance imaging equipment. The metal labels may be paramagnetic, superparamagnetic, or ferromagnetic, or any other metal effective in accordance with the present invention and which can be affectively detected using standard magnetic resonance imaging techniques.

During therapeutic use, using zinc fingers having radioactive metals such as ⁶⁹Zn ⁴⁷SC, ⁶⁷Cu, ¹⁵³Sm, ¹⁰⁵Rh ¹⁸⁸Re, and ¹⁸⁶Re bound thereto will accumulate in the necrotic core and irradiate the viable tumor cells with beta radiation from inside the tumor thereby killing the tumor cells while sparing normal tissues.

In general, the metal is medically-useful metal chosen from the group of metal ions including iron, cobalt, nickel, copper, zinc, arsenic, selenium, technetium, ruthenium, palladium, silver, cadmium, indium, antimony, rhenium, osmium, iridium, platinum, gold mercury, thallium, lead, bismuth, polonium and astatine and may include radionuclides of indium, gold, silver, mercury, technetium, rhenium, copper and ruthenium. In particular, the radionuclides may be ⁶²Cu, ⁶⁴Cu, ⁶⁷Cu, ⁹⁷Ru, ¹⁰⁵Rh, ¹⁰⁹Pd, ¹⁸⁶Re, ¹⁸⁸Re, ¹⁹⁸Au, ¹⁹⁹Au, ²⁰³ Pb, ²¹¹Pb, ²¹²Bi, and ^{99m}Tc. Particularly, useful metal ions can be found in the group consisting of elements 26-30 (Fe, Co, Ni, Cu, Zn), 33-34 (As, Se), 42-50 (Mo, Tc, Ru, Rh, Pd, Ag, Cd, In, Sn) and 75-85 (Re, Os, Ir, Pt, Au, Hg, Tl, Pb, Bi, Po, At). Isotopes of the elements Tc, Re, and Ru are particularly applicable for use in diagnostic imaging and radiotherapy.

A generalized preferred version of a modified peptide for use in the present invention has the following structure:

(AA₁₋₁₀-X₁-AA₁₋₄-X₂-AA₄₋₂₀-X₃-AA₁₋₅-X₄-AA₁₋₁₀)ₙ I

This generalized Formula I represents a zinc-finger peptide backbone having from 1 to 10 amino acids (AA₁₋₁₀) at its N-terminal end linked to a cysteine or histidine residue represented by X₁, which is linked to from one to four amino acids (AA₁₋₄), which are in turn linked to a second cysteine or histidine residue represented by X₂, which in turned is linked to from 4 to 20 amino acids represented by AA₄₋₂₀, which in turn are linked to a third cysteine or histidine residue represented by X₃, which in turn is linked to from one to five amino acids (AA₁₋₅) which in turn are linked to a fourth cysteine or histidine residue (X₄) which is then linked to from one to ten amino acids (AA₁₋₁₀) at the carboxy terminal. This sequence may be repeated one to ten times (n=1-10). At least two of the residues represented in Formula I by an “X” must be cysteine. Alternatively, Formula I may comprise three cysteines, and one histidine, or alternatively, four cysteines and no histidine residues. When Formula I has two or more repeating subunits (i.e., n=2-10), the amino acids in each subunit may be the same, or may be different, in the alternating subunits. Any amino acids may comprise the Formula I as long as the resulting peptide has a tertiary structure which can bind both to a metal and to DNA and/or RNA in accordance with the present invention. Examples of zinc finger peptides which will function in accordance with the present invention are found in A. Travers, DNA-Protein Interactions, London, Chapman & Hall, 1993; Branden et al., Introduction to Protein Structure, New York and London, Garland Publishing, Inc., 1991; K. Struhl, “Helix-Turn-Helix, Zinc-Finger, and Leucine-Zipper Motifs for Eukaryotic Transcriptional Regulatory Proteins”, Trends Biochem. Sci., 14(4) :137-40, 1989; Gibson et al., “A Model for the Tertiary Structure of 28 Residue DNA-Binding Motif (Zinc Finger) Common to Many Eukaryotic Transcriptional Regulatory Proteins”, Protein Eng., 2(3) :209-218, 1988, each of which is hereby incorporated by reference herein.

### Synthesis of Metal-Labeled Zinc Fingers and Zinc Finger Analogues

Methods of labeling (substituting) zinc fingers and their analogues with metals will now be described.

### EXAMPLE 1

### Incorporation of Radioactive Metals

The addition of radioactive metals to zinc fingers can be easily accomplished by one of ordinary skill in the art. For example, radioactive technetium can be incorporated into a zinc finger by the addition of Na ^{99m}TcO₄ to a quantity of unlabeled peptide in the presence of a reducing agent such as stannous chloride, preferably at a pH less than about 8.0 but greater than about 5.0. In an oxidative environment two cysteine residues can be oxidized to form a disulfide bridge. This can occur both between two cysteines in the same molecule or between the cysteines of two different molecules. The formation of a disulfide bridge between two cysteines in the zinc finger peptide will effect the chelation of the metal to the peptide. Since a reducing agent such as stannous chloride is used to reduce the sodium pertechnetate to a reactive species it will also reduce disulfide bridges but at a slower rate. Other reducing agents may comprise other forms of stannous compounds and may include stannous glucoheptonate, stannous gluconate, stannous phosphonate, and stannous fluoride, for example. A variety of dicarboxylic acids can be added to the Sn (II) agent, including phthalate, tartrate or citrate. The thiolate-containing peptide can be in a solution which includes free amino acids, such as glycine. Any effective reducing agent may be used herein.

### EXAMPLE 2

### Radiometal Incorporation into a Zinc Finger Analogue

The following peptide (SEQ ID NO:1) of the Cys-Cys-His-His family was synthesized and used herein to evaluate the concept of zinc finger analogues labeled with ^{99m}Tc. (See Table 1 for identity for the residues listed in SEQ ID NO:1).

| Tyr Gln Cys Glu Ile Cys Gly Lys Ser Phe Ser Asp Lys Ser Asn Leu Thr | SEQ ID NO:1 |
|---|---|
| 1 5 10 15 | |
| Arg His Leu Arg Ile His Thr Gly | |
| 20 25 | |

The peptide having SEQ ID NO:1 was radiolabeled with ^{99m}Tc by the following procedure. One mL of 0.9% sodium chloride (for injection, U.S.P., Abbott Laboratories North Chicago, Ill. 60064, USA N.C. 0074-4888-20) was added to a vial of TECHNESCAN™ Gluceptate. 0.8 mL of the gluceptate solution was removed and the vial was placed in a lead shielding container. To this vial of gluceptate was added a solution of 1 mg of the peptide SEQ ID NO:1 suspended in 0.5 mL of 0.9% sodium chloride (for injection, U.S.P.). Within about two minutes (but not less than one minute) was added 30 mCi of sodium pertechnetate in approximately 0.5 mL. The Na ^{99m}TcO₄ was eluted from a commercial ⁹⁹Mo/^{99m}Tc generator (Dupont, Inc.) one hour previously. The reaction vial was inverted slowly for 30 seconds and allowed to remain at room temperature for one hour, resulting in formation of a composition comprising the peptide SEQ ID NO:1 labeled with ^{99m}Tc.

The ^{99m}Tc radiolabeled zinc finger analogue was analyzed by placing 5 uL of the reaction mixture containing the radiolabeled peptide SEQ ID NO:1 onto a 1×10 cm strip of ITLC-SG (Eastman Kodak Company, Rochester, N.Y. 14650, Cat. # 13179) one cm from the bottom and developing the chromatography with 0.9% sodium chloride (for injection, U.S.P.) allowing the solvent front to migrate 4.5 cm. The chromatogram was analyzed to determine the location of the radioactivity by a solid NaI scintillation detector. About 99.4% of the radioactivity was located at the origin while 0.6% was located at the solvent front. By comparison, ^{99m}Tc-gluceptate when analyzed by the same system migrates with the solvent front and no activity is located at the origin. This indicates that greater than 99% of the added ^{99m}Tc was bound to the synthesized zinc finger analogue (SEQ ID NO:1).

### EXAMPLE 3

### Binding of Radiolabeled Zinc Finger Analogue to Cancer Cells

Utilizing the ^{99m}Tc radiolabeled zinc finger analogue synthesized above (SEQ ID NO:1 labeled with ^{99m}Tc) RAJI and CEM cells were used to evaluate the uptake of the compound in vitro. The RAJI line of lymphoblast-like cells was established from a Burkett's lymphoma. CEM is a T lymphoblastoid cell line from acute lymphoblastic leukemia. Both cell lines were cultured in serum-free/protein-free hybridoma medium 90% and fetal bovine serum 10%. Both cell lines were grown, formalin fixed, ethanol (70%)/acetone fixed, or used as live cells for the experiment. Table 1 shows that live cells with intact membranes substantially failed to take up the analogue while cells fixed with formalin and ethanol (70%) /acetone (i.e., dead cells) absorbed substantial quantities of the analogue. All experiments were conducted in triplicate and the results are averages of the three measurements. The experiment in brief was a follows. Each triplicate set of cells were adjusted to contain about 2 million cells per tube. To each tube was added the freshly prepared ^{99m}Tc radiolabeled zinc finger analogue described above in Example 2 at a concentration of 0.5 ug/10 μCi and the tubes were incubated for one hour on a rotating platform. The original counts were determined in an auto gamma counter and the tubes centrifuged, the supernate removed and washed three times with PBS with 1.0% BSA. At this point the final counts were obtained on the rinsed cells.

TABLE 1

| | | FINAL | % |
|---|---|---|---|
| CELLS | ORIGINAL COUNTS | COUNTS | UPTAKE |
| Formalin Fixed RAJI | 709,919 | 182,412 | 25.7% |
| Formalin Fixed CEM | 718,922 | 156,918 | 21.8% |
| Ethanol/Acetone RAJI | 679,545 | 369,545 | 54.4% |
| Live RAJI | 1,128,191 | 13,851 | 1.2% |

### EXAMPLE 4

### Diagnostic and Therapeutic Kits

The peptide disclosed herein may be provided as a part of a kit for diagnostic or therapeutic use. The peptide may be stored frozen in bulk form after disulfide bond reduction and the removal of excess reducing agent. Alternatively, the peptide may be stored in bulk form or in unit dose form after addition of the Sn (II). Similarly, the peptide may be stored lyophilized during or after processing. For example, in one embodiment the peptide is stored in vials after introduction of the Sn (II). Methods used in lyophilization of peptides are known to those skilled in the art. Either frozen or lyophilized preparations may be maintained for an indefinite period before labeling by the addition of the medically useful metal ion.

In both the frozen and lyophilized storage forms, excipients may be added to the peptide to minimize damage which can arise from ice-crystal formation or free-radical formation. The type of excipient and the concentration depends on the nature of the peptide and the intended use. In one embodiment, glycine and inositol are used as excipients in lyophilized preparations.

A typical lyophilized preparation made by the embodiments set for the above would, upon rehydration, contain approximately 10 mM tartrate, 40 mM phthalate, 22 μg of Sn (II), 500 μg of peptide, 2 mg/ml of glycine, and 2 mg/ml of inositol. The amounts of peptide and Sn (II) or other reducing agent used in the kit would depend on the medical application, varying depending on biodistribution of the peptide, imaging modality being used, type of metal ion and related factors. Similarly, the amount and type of buffer components (such as tartrate and phthalate) and excipients (such as glycine and inositol) depends on the specific application.

To label with a medically useful metal ion, a typical lyophilized preparation is hydrated by the addition of solution containing 0.9% NaCl (U.S.P.) or water for injection (U.S.P.) and the medically useful metal ion. Alternatively, it is possible to hydrate the lyophilized preparation, and to add the metal ion in a subsequent step. If a frozen preparation is used, it is thawed and allowed to come to room temperature, and a solution containing the medically useful metal ion is then added. The nature and amount of the medically useful metal ion and the specific reaction conditions depend on the isotopic nature of the metal, and the intended medical application. In one embodiment, ^{99m}Tc is added in the form of pertechnetate ion in a solution of 0.9% NaCl. The ^{99m}Tc is typically incubated for up to 30 minutes to insure completion of the reaction with the peptide, after which the radio labeled preparation can be directly used in medical applications. In another embodiment, ⁶⁷Cu is added in a solution of 10 mM tartrate and 40 mM phthalate at pH 5.6. In yet another embodiment, ¹⁸⁸Re or ¹⁸⁶Re is added to a solution of 10 mM tartrate and 40 mM phthalate, at pH 5.6, and containing Sn (II), and then heated to lower the oxidation state of Re. The resulting solution is then added to the lyophilized or frozen preparation.

In the embodiment in which ^{99m}Tc is used, the Sn (II) is present in the peptide-containing solution in sufficient excess to alter the oxidation state of the Tc ion such that it can bind to thiolate groups. Typically Tc (VII) is reduced to Tc (III), Tc (VI), and/or Tc (V). The preferred state of Tc to be added to peptide preparations is as the pertechnetate ion, (TcO₄)—. The Sn (II) then reacts with the pertechnetate ion resulting in a lower oxidation state in which the Tc is reactive with thiolate groups. Similar approaches may be used to lower the oxidation sate of other medically useful metal ions for subsequent binding to thiolate groups. The type of the metal ion, its isotopic nature, and concentration would depend on the intended medical application.

### EXAMPLE 5

### Technetium Tc-99m Labeling Kit

Each 10 mL vial contains one mg zinc finger peptide analogue, between 0.05 mg stannous chloride dihydrate (SnCl₂.2H₂O) and 0.2 mg total tin expressed as stannous chloride dihydrate, 40 mg sodium tartrate dihydrate (Na₂C₄H₂O₆.2H₂O) and 20 mg lactose monohydrate. Prior to lyophilization the pH may be adjusted sodium hydroxide or hydrochloric acid. The pH of the reconstituted radiopharmaceutical is between 5.0 and 6.0. The vial will contain argon or nitrogen in the head space. The Tc-99m radiolabeled zinc finger peptide analogue is produced by the addition of 4 to 10 mL of sodium pertechnetate Tc-99m solution containing 20 mCi (740 megabecquerels) to 100 mCi (3.7 gigabecquerels) into the vial. The vial is inverted for 30 seconds and allowed to stand for one hour or alternatively placed the reaction vial briefly in a boiling water bath.

### EXAMPLE 6

### Indium In-111 Labeling Kit

Each kit consist of two vials containing all of the non-radioactive components necessary to produce a single dose of Indium In-111 zinc finger peptide analogue for administration by intravenous injection. The zinc finger peptide analogue vial contains 0.5 mg of zinc finger peptide analogue in one mL of sodium phosphate buffered saline solution adjusted to pH 6. A second vial of sodium acetate buffer contains 82 mg of sodium acetate in two mL of Water for Injection adjusted to pH 5-7 with glacial acetic acid. Both vials are sterile, pyrogen-free, clear, colorless solutions. The sodium acetate buffer solution must be added to sterile, non-pyrogenic high purity Indium In-111 Chloride solution 5 mCi (185 megabecquerels) to 10 mCi (370 megabecquerels) to buffer it prior to radiolabeling the zinc finger peptide analogue. Once buffered, the entire contents of this vial is added to the vial containing the zinc finger peptide analogue and this results in incorporation of the Indium In-111 into the peptide and the formation of the nucleic acid binding metal-peptide complex.

### Utility

The present invention provides a method for the diagnosis of a patient suspected of being afflicted with diseases characterized by foci of necrotic tissue such myocardial diseases, and primary and metastatic cancers, or any other disease characterized by damaged or necrotic tissues. The present invention further provides a method of treating a patient having primary tumor cancer or metastatic cancer by treating the patient with an effective amount of a compound of the present invention.

An effective amount of a compound of the present invention refers to an amount which is effective in enabling diagnosis or treatment of a disease condition contemplated herein. Where used as treatment, treatment refers to an effort to control the disease.

The term “controlling” is intended to refer to all processes wherein there may be a slowing, interrupting, arresting, or stopping of the progression of the disease and does not necessarily indicate a total elimination of all disease symptoms. Where used herein, the term “purified” zinc finger refers to a zinc finger compound which is substantially free of natural contaminants.

The term “therapeutically effective amount” is further meant to define an amount resulting in the improvement of any parameters or clinical symptoms characteristic of a cancerous condition. Actual doses used for treatment and diagnosis will vary for the various specific molecules contemplated as being covered by the present invention, and will vary with the patient's overall condition, the seriousness of the symptoms, and counterindications.

As used herein, the term “subject” or “patient” refers to a warm blooded animal such as a mammal which is afflicted with a particular disease state. It is understood that guinea pigs, dogs, cats, rats, mice, rabbits, horses, cattle, sheep, and humans and other primates are examples of animals within the scope of the meaning of the term.

A therapeutically effective amount of the compound used in the treatment described herein can be readily determined by the attending diagnostician, as one skilled in the art, by the use of conventional techniques and by observing results obtained under analogous circumstances. In determining the therapeutically effective dose, a number of factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailability characteristic of the preparation administered; the dose regimen selected; the use of concomitant medication; and other relevant circumstances.

A therapeutically or diagnostically effective amount of the composition of the present invention will generally be a dose containing sufficient active ingredient to deliver from about 5 mCi to about 1000 mCi (curies active ingredient/standard man of approximately 70 kg) when the conjugated metal is a radiometal. Preferably, the composition will deliver at least 10 mCi to 50 mCi.

A diagnostically effective dosage of the composition of the present invention when the metal is paramagnetic, superparamagnetic, or ferromagnetic is from 1 μmol/kg of body weight to 1.0 mmol/kg of body weight, and more particularly an amount which can be readily determined by a person of ordinary skill in the art using standard medical techniques.

Practice of the method of the present invention comprises administering to a patient a therapeutically or diagnostically effective amount of the active ingredient(s), in any suitable systemic or local formulation, in an amount effective to deliver the dosages listed above. The dosage can be administered on a one-time basis, or (for example) from one to 5 times per day, depending on the patient condition.

Preferred amounts and modes of administration are able to be determined by one skilled in the art. One skilled in the art of preparing formulations can readily select the proper form and mode of administration depending upon the particular characteristics of the compound selected, the disease state to be treated, the stage of the disease, and other relevant circumstances using formulation technology known in the art, described for example in Remington's Pharmaceutical Sciences, latest edition, Mack Publishing Co.

Pharmaceutical compositions can be manufactured utilizing techniques known in the art and as described elsewhere herein. Typically the therapeutically or diagnostically effective amount of the compound will be admixed with a pharmaceutically acceptable carrier such as a saline solution for internal administration to the subject.

The compounds or compositions of the present invention may be administered by a variety of routes including parenterally (i.e. subcutaneously, intravenously, intramuscularly, intraperitoneally, or intratracheally).

For parenteral administration the compounds may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives, and buffers as are known in the art.

As noted above, the compositions can also include an appropriate carrier. For surgical implantation, the active ingredients may be combined with any of the well-known biodegradable and bioerodible carriers, such as polylactic acid and collagen formulations. Such materials may be in the form of solid implants, sutures, sponges, wound dressings, and the like. In any event, for local use of the materials, the active ingredients usually be present in the carrier or excipient in a weight ratio of from about 1:1000 to 1:20,000, but are not limited to ratios within this range. Preparation of compositions for local use are detailed in Remington's Pharmaceutical Sciences, latest edition, (Mack Publishing).

Additional pharmaceutical methods may be employed to control the duration of action. Controlled release preparations: may be achieved through the use of polymers to complex or absorb the active ingredient. The controlled delivery may be achieved by selecting appropriate macromolecules (for example, polyesters, polyamino acids, polyvinyl, pyrrolidone, ethylenevinylacetate, methylcellulose, carboxymethylcellulose, orprotamine, sulfate) and the appropriate concentration of macromolecules as well as the methods of incorporation, in order to control release.

Another possible method useful in controlling the duration of action by controlled release preparations is incorporation of the active ingredient into particles of a polymeric material such as polyesters, polyamino acids, hydrogels, poly(lactic acid), or ethylene vinylacetate copolymers.

Alternatively, instead of incorporating the active ingredient into polymeric particles, it is possible to entrap these materials in microcapsules prepared, for example, by coacervation techniques or by interfacial polymerization (for example, hydroxymethylcellulose or gelatine-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are disclosed in the latest edition of Remington's Pharmaceutical Sciences.

U.S. Pat. No. 4,789,734 describe methods for encapsulating biological materials in liposomes. Essentially, the material is dissolved in an aqueous solution, the appropriate phospholipids and lipids added, along with surfactants if required, and the material dialyzed or sonicated, as necessary. A good review of known methods is by G. Gregoriadis, Chapter 14. “Liposomes”, Drug Carriers in Biology and Medicine, pp. 287-341 (Academic Press, 1979). Microspheres formed of polymers or proteins are well known to those skilled in the art, and can be tailored for passage through the gastrointestinal tract directly into the blood stream. Alternatively, the agents can be incorporated and the microspheres, or composite of microspheres, implanted for slow release over a period of time, ranging from days to months.

When the composition is to be used as an injectable material, it can be formulated into a conventional injectable carrier. Suitable carriers include biocompatible and pharmaceutically acceptable phosphate buffered saline solutions, which are preferably isotonic.

The zinc finger peptides of the present invention may comprise additional chemical moieties not normally a part of the molecule. Such moieties may improve the molecule's solubility, absorption, and biological half-life. The moieties may alternatively decrease the toxicity of the molecule, eliminate or attenuate any undesirable side effect of the molecule. Examples of moieties capable of mediating such effects are disclosed in the latest edition of Remington's Pharmaceutical Sciences, and will be apparent to those of ordinary skill in the art.

The metal conjugated peptides of the present invention and functional derivatives can be formulated according to known methods of preparing pharmaceutically useful compositions, whereby these materials or their functional derivatives are combined in a mixture with a pharmaceutically acceptable carrier vehicle. Suitable vehicles and their formulation, including other human proteins, e.g., human serum albumin, are described, for example, in Remington's Pharmaceutical Sciences, (Mack Publishing Co., 1980).

For reconstitution of a lyophilized product in accordance with this invention, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which fare available to those in the field.

The pharmaceutical composition may also be in the form of an aqueous solution containing many of the same substances as described above for the reconstitution of a lyophilized product.

The compounds can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

As mentioned above, the products of the invention may be incorporated into pharmaceutical preparations which may be used for therapeutic purposes. However, the term “pharmaceutical preparation” is intended in a broader sense herein to include preparations containing a protein composition in accordance with this invention, used not only for therapeutic purposes but also for reagent or diagnostic purposes as known in the art, or for tissue culture. The pharmaceutical preparation intended for therapeutic use should contain a “pharmaceutically acceptable” or “therapeutically effective amount” of the metal conjugated peptide, i.e., that amount necessary for preventative or curative health measures. If the pharmaceutical preparation is to be employed as a reagent or diagnostic, then it should contain reagent or diagnostic amounts of the compound.

As noted above, the invention contemplated herein comprises a pharmaceutical composition for in vivo or in vitro diagnosis of cancer, myocardial damage, or other pathological conditions characterized by the presence of necrotic tissues in the diseased tissues. In one embodiment, the pharmaceutical composition comprises a compound comprising a peptide and a radioactive metal bound thereto (the peptide having a tertiary conformation capable of binding to mammalian DNA and/or RNA). The cancers diagnosed may be primary tumors or metastases. In one diagnostic method, an effective amount of the compound is administered to an individual suspected of having malignant tissues. The compound is delivered by vascular tissue to the site of the malignant tissues where the compound binds preferentially to the DNA of necrotic malignant cells of primary and/or metastatic cancer tissues. As noted above, the compound binds to the DNA of necrotic malignant tissues because the internal cellular components of the necrotic malignant cells are no longer enclosed within an intact cellular membrane, unlike healthy living cells. Once the compound has sufficiently bound to components within the necrotic tissues, the sites of the affected tissues within the body can be imaged using standard nuclear medicine imaging techniques well known to those of ordinary skill in the art.

Similarly, when the compound has a paramagnetic, superparamagnetic, or ferromagnetic metal bound thereto, the same methods can be used, except the imaging techniques are standard magnetic resonance imaging techniques well known to those of ordinary skill in the art. The same methods can be used in the diagnosis of myocardial (heart) disease using standard nuclear and/or magnetic resonance imaging techniques, wherein the compound binds to the internal cellular components of damaged and degenerating cells of the myocardium. The detection imaging may comprise imaging with at least one method selected from the group consisting of gamma scintigraphy, specific photon emission computerized tomography, positron emission tomography and magnetic resonance imaging.

Changes may be made in the construction and the operation of the various components, elements and kits described herein or in the steps or the sequence of steps of the methods described herein without departing from the spirit and scope of the invention as defined in the following claims.

What is claimed:

1. A compound comprising:
a metal-peptide complex formed by the chelation of a metal to a zinc finger peptide, the metal-peptide complex having a tertiary structure enabling said metal-peptide complex to bind to a mammalian nucleic acid, wherein the metal is not zinc, iron, cadmium or cobalt.

2. The compound of claim 1 wherein the zinc finger peptide has a metal binding site comprising amino acid residues selected from the group consisting of four cysteine residues, one histidine and three cysteine residues, and two cysteine and two histidine residues to which the metal is complexed.

3. The compound of claim 1 wherein the metal is selected from the group consisting of ionic forms of nickel, copper, arsenic, selenium, gadolinium, technetium, ruthenium, palladium, silver, indium, antimony, rhenium, osmium, iridium, platinum, gold, mercury, thallium, lead, bismuth, polonium, astatine, manganese, indium, samarium and scandium.

4. The compound of claim 1 wherein the radionuclide is selected from the group consisting of ⁶²Cu, ⁶⁴Cu, ⁶⁷Cu, ⁹⁷Ru, ¹⁰⁵Rh, ¹⁰⁹Pd, ¹⁸⁶Re, ¹⁸⁸Re, ¹⁹⁸Au, ¹⁹⁹Au, ²⁰³Pb, ²¹¹Pb, ²¹²Bi, ^{99m}Tc, and ¹¹¹In.

5. A composition comprising the metal-peptide complex of claim 1 disposed within a pharmaceutically acceptable carrier.

6. A compound comprising:
a metal-peptide complex formed by the chelation of a metal atom to a zinc finger peptide, the metal-peptide complex having a tertiary structure enabling said metal-peptide complex to bind to a mammalian nucleic acid, wherein the metal is selected from the group consisting of indium, technetium, rhenium and ruthenium.

7. A compound comprising:
a radionuclide-peptide complex formed by the chelation of a radionuclide to a zinc finger peptide, the radionuclide-peptide complex having a tertiary structure enabling said radionuclide-peptide complex to bind to a mammalian nucleic acid, wherein the radionuclide is not zinc, iron, cadmium or cobalt.

8. A method of determining the location of diseased tissues in a mammal, the diseased tissues characterized by the presence of degenerating neoplastic or myocardial cells, comprising:
providing a quantity of a metal-peptide complex formed by the chelation of a metal atom to a zinc finger peptide, the metal-peptide complex having a tertiary structure enabling said metal-peptide complex to bind to a mammalian nucleic acid;
administering to the mammal the metal-peptide complex in an amount effective for imaging wherein the metal-peptide complex is deposited in degenerating cells of the diseased tissues and preferentially binds to nucleic acids therein forming a detectable deposition site; and
imaging the deposition site by metal ion detection means.

9. The method of claim 8 wherein the degenerating neoplastic cells are cells in a primary cancer tumor or in a metastatic cancer.

10. The method of claim 8 wherein in the imaging step the metal ion detection means are magnetic resonance imaging means or nuclear medicine imaging means.

11. The method of claim 8 wherein the metal-peptide complex comprises a metal selected from the group consisting of ionic forms of nickel, copper, arsenic, selenium, gadolinium, technetium, ruthenium, palladium, silver, indium, antimony, rhenium, osmium, iridium, platinum, gold, mercury, thallium, lead, bismuth, polonium, astatine, manganese, indium, samarium and scandium.

12. The method of claim 8 wherein the metal of the metal-peptide complex comprises a radioisotope.

13. The method of claim 12 wherein the metal-peptide complex comprises a metal selected from the group of radioisotopes consisting of radioisotopes of indium, technetium, rhenium and ruthenium.

14. The method of claim 8 wherein in the administering step, the effective amount of the metal-peptide complex comprises from about 5 to about 1000 mCi.

15. The method of claim 8 wherein in the administering step, the effective amount of a paramagnetic, superparamagnetic, or ferromagnetic metal in the metal-peptide complex is 1 μmol/kg of mammal weight to 1.0 mmol/kg of mammal weight.

16. The method of claim 8 wherein the metal-peptide complex is administered parenterally.

17. The method of claim 8 wherein the metal-peptide complexed is administered as a composition comprising a pharmaceutically acceptable carrier.

18. A therapeutic treatment for killing neoplastic cancer cells in a primary tumor or a metastatic tumor in a mammal, comprising:
providing a metal-peptide complex formed by the chelation of a metal atom to a zinc finger peptide, the metal-peptide complex having a tertiary structure enabling said metal-peptide complex to bind to a mammalian nucleic acid, wherein the metal is a radiometal; and
administering to the mammal an amount of the metal-peptide complex wherein the metal-peptide complex is deposited in necrotic neoplastic cells in the primary tumor or metastatic tumor and preferentially binds to nucleic acids in said necrotic cells in an amount sufficient to deliver a lethal amount of radiation to living neoplastic cells in the vicinity of the necrotic neoplastic cells near the metal-peptide complex therein.

19. The method of claim 18 wherein the metal-peptide complex comprises a metal selected from the group consisting of radioisotopes of indium, technetium, rhenium and ruthenium.

20. The method of claim 18 wherein in the administering step, the effective amount of the metal-peptide complex comprises from about 5 to about 1000 mCi.

21. A kit for use in preparing a radiopharmaceutical composition, comprising:
a container having an amount of a zinc finger peptide disposed therein; and
a reducing agent for reducing the zinc finger peptide to prepare the peptide for complexing with a radiometal to form a metal-peptide complex having a tertiary structure enabling said metal-peptide complex to bind to a mammalian nucleic acid.

22. The kit of claim 21 further comprising a radiometal for complexing to the peptide for forming the metal-peptide complex.

23. The kit of claim 21 wherein the metal is selected from the group consisting of ionic forms of nickel, copper, arsenic, selenium, gadolinium, technetium, ruthenium, palladium, silver, indium, antimony, rhenium, osmium, iridium, platinum, gold, mercury, thallium, lead, bismuth, polonium, astatine, manganese, indium, samarium and scandium.

24. The kit of claim 21 wherein the metal is selected from the group consisting of radioisotopes indium, technetium., rhenium and ruthenium.

25. The kit of claim 24 wherein the reducing agent comprises an amount of stannous ion in the form of stannous glucoheptonate, stannous gluconate, stannous phosphonate, stannous chloride, and stannous fluoride.
